(19) United States
(12) Patent Application Publication
(10) Pub. No.: US 2013/0288989 A1
(43) Pub. Date: Oct. 31, 2013

(54) NEW USE OF CHEMICAL INGREDIENTS IN CYNOMORIUM AS PHYTOESTROGEN

(71) Applicant: Xiumei Gao, Tianjin (CN)

(72) Inventor: Xiumei Gao, Tianjin (CN)

(73) Assignee: TIANJIN UNIVERSITY OF TRADITIONAL CHINESE MEDICINE, TIANJIN (CN)

(21) Appl. No.: 13/820,249

(22) PCT Filed: Oct. 14, 2010
(86) PCT No.: PCT/CN2010/001611

§ 371 Date: Mar. 1, 2013

(51) Int. Cl. A61K 31/7048; A61K 31/353; A61K 31/122; A61K 31/7034

(52) U.S. Cl. CPC A61K 31/7048; A61K 31/7034; A61K 31/353; A61K 31/122
USPC 514/27; 552/262; 514/680; 552/261; 549/399; 514/456; 536/18.1; 536/4.1; 514/25

(57) ABSTRACT

Use of chemical ingredients in Cynomorium as phytoestrogen is provided by the present invention. Particularly, use of Cynomorium, extracts of Cynomorium, or at least one ingredient chosen from chrysophanol, emodin, catechin, (−)-epicatechin-3-O-gallate, naringenin-4′-O-glucopyranoside, and phlorizin, in manufacturing medicaments for treating and/or preventing diseases related to insufficient of estrogen secretion in mammal (for example, human), or use in manufacturing medicaments used as phytoestrogens is provided by the present invention. The extracts of Cynomorium, compositions containing extracts of Cynomorium or active ingredients thereof, and methods of treating and/or preventing diseases related to insufficient of estrogen secretion are also provided by the present invention. According to the present invention, Cynomorium, extracts of Cynomorium, active ingredients thereof may be used to treat and/or prevent menopausal syndrome, osteoporosis, degenerative osteoporosis, cardiovascular and cerebrovascular diseases and breast cancer.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The present invention relates to new use of several chemical ingredients from traditional Chinese medicine Herba Cynomorii, and particularly to new use of flavone compounds and anthraquinone compounds from Herba Cynomorii as phytoestrogen.

Herba Cynomorii is the dry chylocaulous of Chynomorium songaricum Rupr. Herba Cynomorii has sweet flavor and warm nature, belongs to liver, kidney and large intestine meridians, and is an unique rare natural psammophyte. It has functions of reinforcing kidney Yang, benefiting blood and essence, loosening bowel and relieving constipation, and may be used for the treatment of impotence and seminal emission, soreness and weakness of waist and knees, dryness of intestine and constipation, blood exhaustion, hematuria, neurasthenia, etc. The studying of chemical ingredients of Herba Cynomorii indicates Herba Cynomorii contains flavones, triterpenes, saccharides and glycosides, tannins, steroids, volatile ingredients, organic acids, amino acids, inorganic elements and ions, in which the flavones include (+)-catechin, (−)-catechin, naringenin 4′-O-glycopyranoside, and naringenin is glycoside of aglycon.

Estrogens are critical regulatory hormones for female, which are mainly produced by ovary and placenta, and estrogens in their chemical structure belong to steroid hormones, have liposolubility and many target tissues including reproductive system, bones, cardiovascular. Estrogens generally are divided into two groups: steroid estrogens and non-steroid estrogens. Steroid estrogens are endogenous ingredients secreted by ovary of mammals. So far, three steroid estrogens are found in human body: estradiol, estriol separated from urine of gravida, and estrone, in which estradiol has the strongest activity. Steroid estrogens have important functions in sustaining life, regulating sexual function, physical development, immune-regulation, treatment of skin diseases and procreation. Non-steroid estrogens mainly include stilbene glycoside compounds. It is generally believed that estrogens generate physiological effects by binding to corresponding estrogen receptors (ER) in cells, thereby inducing a series of downstream events such as inducing and starting transcription. The affinity of non-steroid estrogens to estrogen receptor is 1/100 or 1/1000 of that of estradiol. Since the long-term clinical use of estrogens may result in breast cancer, endometriosis and carcinogenicity, the alternates of estrogens are in request. Phytoestrogens (PE) are non-steroid compounds generally existing in plants, and may bind to in vivo ER, and epidemiologic studies and animal tests confirm phytoestrogens have good prophylactic and therapeutical effects in prevention of tumors and treatment of cardiovascular diseases and osteoporosis, improvement of menopausal symptom, may protect nervous system and show none of the above adverse effects. Hence, phytoestrogen are considered as alternates of natural estrogens. Although the affinity of PE to estrogen receptor is only 1/500- 1/1000 of that of estrogens, PE has less side-effect in hormone replacement therapy (HRT), and draws huge attentions from researchers and is promising in HRT.

Estrogens have a wide scope of physical functions in growth, development, cell differentiation and reproductive system in organisms. Estrogens bring into various biological effects mainly through ER. There are two subtypes of ER, ERα and ERβ, which have different expression levels in many organs and cells in human body. The two subtypes have difference in expression level in the cells of same one tissue, and the same subtype has differences in expression level in the cells of difference tissues. Hence, the effects of interaction between any ligand and ERα and ERβ are complicated. ERα and ERβ have difference in tissue distribution, in which uterus, mammary gland, oviduct, vaginal epithelium mainly express ERα and have weak expression of ERβ, while ovary has predominant expression of ERβ, most existing in granular cells, and vascular smooth muscle cells mainly express ERβ.

Women in climacteric period have depleted ovary, reduced estrogen secretion, and thus various physiological processes mediated by estrogens are influenced, thereby bringing about physiological and psychological symptoms such as palpitation, oppression in chest, apprehension, depression, easy excitation, insomnia, hypomnesis, these symptoms are generally called as climacteric syndrome; the osteoporosis caused by the reduction of estrogen secretion is called degenerative osteoporosis. In order to treat and/or prevent the above disorders, one beneficial method is to replenish estrogens. However, it is found in many years of clinical practices that the risk of breast cancer attack increases greatly after long-term administration of estrogens. Hence, it is still desired by those skilled in the art to find phytoestrogens that have an activity of estrogens and/or no side-effects of estrogens, so as to provide a new therapeutical option for clinical application.

### BRIEF SUMMARY

The object of the present invention is to provide phytoestrogens that have an activity of estrogens and/or no side-effects of estrogens, so as to provide a new therapeutical option for clinical application. The inventors of the present invention has surprisingly found that chemical ingredients, such as flavone and anthraquinone compounds, extracted from Chinese medicine Herba Cynomorii, have established activities of estrogens, and may be useful in the estrogen replacement therapy as phytoestrogens. The present invention has been accomplished on the basis of the above finding.

The first aspect of the present invention provides use of Herba Cynomorii in the manufacture of a medicament for the treatment and/or prophylaxis of a disease associated with insufficient secretion of estrogens in a mammal (e.g., human).

The second aspect of the present invention provides a Herba Cynomorii extract, which comprises at least one of ingredients selected from the group consisting of: chrysophanol, emodin, catechin, (−)epicatechin-3-O-gallate, naringenin-4′-O-glucoside, and phlorizin.

The third aspect of the present invention provides one or more active ingredients selected from the group consisting of: chrysophanol, emodin, catechin, (−)epicatechin-3-β-gallate, naringenin-4′-O-glucoside, and phlorizin, which are obtainable from Herba Cynomorii.

The fourth aspect The fourth aspect of the present invention provides use of a Herba Cynomorii extract in the manufacture of a medicament for the treatment and/or prophylaxis of a disease associated with insufficient secretion of estrogens in a mammal (e.g., human).

The fifth aspect of the present invention provides use of one or more ingredients selected from the group consisting of: chrysophanol, emodin, catechin, (−)epicatechin-3-β-gallate, naringenin-4′-O-glucoside, and phlorizin in the manufacture of a medicament for the treatment and/or prophylaxis of a disease associated with insufficient secretion of estrogens in a mammal (e.g., human).

The sixth aspect of the present invention provides use of Herba Cynomorii in the manufacture of a medicament as phytoestrogen.

The seventh aspect of the present invention provides use of Herba Cynomorii extract in the manufacture of a medicament as phytoestrogen.

The eighth aspect of the present invention provides use of one or more ingredients selected from the group consisting of: chrysophanol, emodin, catechin, (−)epicatechin-3-β-gallate, naringenin-4′-O-glucoside, and phlorizin in the manufacture of a medicament as phytoestrogen.

The ninth aspect of the present invention provides a pharmaceutical composition for the treatment and/or prophylaxis of a disease associated with insufficient secretion of estrogens in a mammal (e.g., human), which comprises a therapeutically and/or prophylactically effective amount of Herba Cynomorii extract and optionally a pharmaceutically acceptable excipient.

The tenth aspect of the present invention provides a pharmaceutical composition for the treatment and/or prophylaxis of a disease associated with insufficient secretion of estrogens in a mammal (e.g., human), which comprises a therapeutically and/or prophylactically effective amount of at least one pharmaceutically active ingredient and optionally a pharmaceutically acceptable excipient, wherein the pharmaceutically active ingredient is selected from chrysophanol, emodin, catechin, (−)epicatechin-3-O-gallate, naringenin-4′-O-glucoside, and phlorizin.

The eleventh aspect of the present invention provides a method for the treatment and/or prophylaxis of a disease associated with insufficient secretion of estrogens in a subject in need thereof, which comprises administering to the subject a therapeutically and/or prophylactically effective amount of Herba Cynomorii, Herba Cynomorii extract or at least one active ingredient selected from the group consisting of: chrysophanol, emodin, catechin, (−) epicatechin-3-O-gallate, naringenin-4′-O-glucoside, and phlorizin.

### DETAILED DESCRIPTION

The above description is given by way of example, and not limitation. Given the above disclosure, one skilled in the art could devise variations that are within the scope and spirit of the invention disclosed herein Further, the various features of the embodiments disclosed herein can be used alone, or in varying combinations with each other and are not intended to be limited to the specific combination described herein. Thus, the scope of the claims is not to be limited by the illustrated embodiments.

The first aspect of the present invention provides use of Herba Cynomorii in the manufacture of a medicament for the treatment and/or prophylaxis of a disease associated with insufficient secretion of estrogens in a mammal (e.g., human).

According to the use any embodiment of the first aspect of the present invention, the disease associated with insufficient secretion of estrogens is selected from the group consisting of: climacteric syndrome, osteoporosis, degenerative osteoporosis, cardiovascular and cerebrovascular diseases, and breast cancer.

According to the use of any embodiment of the first aspect of the present invention, the daily dose of the medicament relative to per kg of body weight of the mammal is equivalent to 0.01-10 g, preferably 0.02-5 g, more preferably 0.05-1 g, and further more preferably 0.05-0.5 g of Herba Cynomorii raw material.

According to the use of any embodiment of the first aspect of the present invention, the medicament comprises an extract from Herba Cynomorii raw material.

According to the use of any embodiment of the first aspect of the present invention, the medicament comprises an extract from Herba Cynomorii raw material obtained by using water, an alcohol (e.g., ethanol), or a water-alcohol (e.g., ethanol) mixture (e.g., 50-98% ethanol, such as 60% ethanol, 95% ethanol) as the extracting solvent.

According to the use of any embodiment of the first aspect of the present invention, the medicament comprises an extract from Herba Cynomorii raw material, and the extract is obtained by extraction using water, an alcohol (e.g., ethanol), or a water-alcohol (e.g., ethanol) mixture (e.g., 50-98% ethanol, such as 60% ethanol, 95% ethanol), and sequentially petroleum ether, chloroform, ethyl acetate, and n-butanol as the solvents.

According to the use of any embodiment of the first aspect of the present invention, the medicament comprises an extract from Herba Cynomorii raw material, which extract is the petroleum ether extract, chloroform extract, ethyl acetate extract, or a mixture thereof, obtained by extraction using water, an alcohol (e.g., ethanol), or a water-alcohol (e.g., ethanol) mixture (e.g., 50-98% ethanol, such as 60% ethanol, 95% ethanol), and sequentially petroleum ether, chloroform, ethyl acetate, and n-butanol as the solvents. In an embodiment, the extract is a mixture of the petroleum ether extract, chloroform extract, and ethyl acetate extract obtained by extraction using water, an alcohol (e.g., ethanol), or a water-alcohol (e.g., ethanol) mixture (e.g., 50-98% ethanol, such as 60% ethanol, 95% ethanol), and sequentially petroleum ether, chloroform, ethyl acetate, and n-butanol as the solvents. In an embodiment, the extract is a mixture of the chloroform extract and ethyl acetate extract obtained by extraction using water, an alcohol (e.g., ethanol), or a water-alcohol (e.g., ethanol) mixture (e.g., 50-98% ethanol, such as 60% ethanol, 95% ethanol), and sequentially petroleum ether, chloroform, ethyl acetate, and n-butanol as the solvents. In an embodiment, the extract is the ethyl acetate extract obtained by extraction using water, an alcohol (e.g., ethanol), or a water-alcohol (e.g., ethanol) mixture (e.g., 50-98% ethanol, such as 60% ethanol, 95% ethanol), and sequentially petroleum ether, chloroform, ethyl acetate, and n-butanol as the solvents. In an embodiment, the extract is the petroleum ether extract obtained by extraction using water, an alcohol (e.g., ethanol), or a water-alcohol (e.g., ethanol) mixture (e.g., 50-98% ethanol, such as 60% ethanol, 95% ethanol), and sequentially petroleum ether, chloroform, ethyl acetate, and n-butanol as the solvents. In an embodiment, the extract is a mixture of the ethyl acetate extract and petroleum ether extract obtained by extraction using water, an alcohol (e.g., ethanol), or a water-alcohol (e.g., ethanol) mixture (e.g., 50-98% ethanol, such as 60% ethanol, 95% ethanol), and sequentially petroleum ether, chloroform, ethyl acetate, and n-butanol as the solvents.

According to the use of any embodiment of the first aspect of the present invention, the medicament comprises an extract from Herba Cynomorii raw material, which extract comprises at least one of the following ingredients selected from the group consisting of: chrysophanol, emodin, catechin, (−)epicatechin-3-O-gallate, naringenin-4′-O-glucoside, and phlorizin. In an embodiment, the extract comprises the following ingredients: chrysophanol, emodin, catechin, (−)epicatechin-3-O-gallate, naringenin-4′-O-glucoside, and phlorizin.

According to the use of any embodiment of the first aspect of the present invention, the medicament comprises an extract from Herba Cynomorii raw material, which extract comprises at least one of the following ingredients selected from the group consisting of: catechin, (−)epicatechin-3-O-gallate, naringenin-4′-O-glucoside, and phlorizin. In an embodiment, the extract comprises the following ingredients: catechin, (−)epicatechin-3-β-gallate, naringenin-4′-O-glucoside, and phlorizin. In an embodiment, the extract is ethyl acetate extract obtained by extraction using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol.

According to the use of any embodiment of the first aspect of the present invention, the medicament comprises an extract from Herba Cynomorii raw material, which extract of Herba Cynomorii is prepared by a process comprising the following steps: extracting Herba Cynomorii with an aqueous ethanol solution (e.g., 50-98% ethanol, such as 60% ethanol, 95% ethanol); extracting the obtained alcohol extract with petroleum ether, and concentrating the ether layer to obtain a petroleum ether portion; extracting the residue of petroleum ether extraction with chloroform, and concentrating the chloroform layer to obtain a chloroform portion; extracting the residue of chloroform extraction with ethyl acetate, and concentrating the ethyl acetate layer to obtain an ethyl acetate portion; extracting the residue of ethyl acetate extraction with n-butanol, and concentrating the n-butanol layer to obtain a n-butanol portion; and combining the above petroleum ether portion, chloroform portion and ethyl acetate portion to obtain the extract. In an embodiment, the Herba Cynomorii extract is obtained by combining the above petroleum portion and chloroform portion. In an embodiment, the Herba Cynomorii extract is obtained by combining the above chloroform portion and ethyl acetate portion. In an embodiment, the Herba Cynomorii extract is obtained by combining the above petroleum ether portion and ethyl acetate portion. In an embodiment, the Herba Cynomorii extract is the above ethyl acetate portion.

The second aspect of the present invention provides a Herba Cynomorii extract, which comprises at least one of ingredients selected from the group consisting of: chrysophanol, emodin, catechin, (−)epicatechin-3-O-gallate, naringenin-4′-O-glucoside, and phlorizin. In an embodiment, the Herba Cynomorii extract comprises chrysophanol, emodin, catechin, (−) epicatechin-3-O-gallate, naringenin-4′-O-glucoside, and phlorizin. In an embodiment, the Herba Cynomorii extract comprises at least one of ingredients selected from the group consisting of: catechin, (−)epicatechin-3-O-gallate, naringenin-4′-O-glucoside, and phlorizin. In an embodiment, the Herba Cynomorii extract comprises catechin, (−)epicatechin-3-O-gallate, naringenin-4′-O-glucoside, and phlorizin.

According to any embodiment of the second aspect, the present invention provides a Herba Cynomorii extract, in which the total amount of chrysophanol, emodin, catechin, (−) epicatechin-3-O-gallate, naringenin-4′-O-glucoside, and phlorizin is 1-90% (w/w), preferably 1-80% (w/w), preferably 2-50% (w/w), preferably 2-40% (w/w), preferably 2-30% (w/w), and more preferably 2-20% (w/w) relative to the total amount of the extract.

According to any embodiment of the second aspect, the present invention provides a Herba Cynomorii extract, which is an extract obtained by extracting Herba Cynomorii raw material with water, an alcohol (e.g., ethanol), or a water-alcohol (e.g., ethanol) mixture (e.g., 50-98% ethanol, such as 60% ethanol, 95% ethanol) as the solvent.

According to any embodiment of the second aspect, the present invention provides a Herba Cynomorii extract, which is an extract obtained by extraction using water, an alcohol (e.g., ethanol), or a water-alcohol (e.g., ethanol) mixture (e.g., 50-98% ethanol, such as 60% ethanol, 95% ethanol), and sequentially petroleum ether, chloroform, ethyl acetate, and n-butanol as the solvents.

According to any embodiment of the second aspect, the present invention provides a Herba Cynomorii extract, which is the petroleum ether extract, chloroform extract, ethyl acetate extract, or a mixture thereof obtained by extraction using water, an alcohol (e.g., ethanol), or a water-alcohol (e.g., ethanol) mixture (e.g., 50-98% ethanol, such as 60% ethanol, 95% ethanol), and sequentially petroleum ether, chloroform, ethyl acetate, and n-butanol as the solvents. In an embodiment, the extract is a mixture of the petroleum ether extract, chloroform extract and ethyl acetate extract obtained by extraction using water, an alcohol (e.g., ethanol), or a water-alcohol (e.g., ethanol) mixture (e.g., 50-98% ethanol, such as 60% ethanol, 95% ethanol), and sequentially petroleum ether, chloroform, ethyl acetate, and n-butanol as the solvents. In an embodiment, the extract is a mixture of the chloroform extract and ethyl acetate extract obtained by extraction using water, an alcohol (e.g., ethanol), or a water-alcohol (e.g., ethanol) mixture (e.g., 50-98% ethanol, such as 60% ethanol, 95% ethanol), and sequentially petroleum ether, chloroform, ethyl acetate, and n-butanol as the solvents. In an embodiment, the extract is the ethyl acetate extract obtained by extraction using water, an alcohol (e.g., ethanol), or a water-alcohol (e.g., ethanol) mixture (e.g., 50-98% ethanol, such as 60% ethanol, 95% ethanol), and sequentially petroleum ether, chloroform, ethyl acetate, and n-butanol as the solvents. In an embodiment, the extract is the petroleum ether extract obtained by extraction using water, an alcohol (e.g., ethanol), or a water-alcohol (e.g., ethanol) mixture (e.g., 50-98% ethanol, such as 60% ethanol, 95% ethanol), and sequentially petroleum ether, chloroform, ethyl acetate, and n-butanol as the solvents. In an embodiment, the extract is a mixture of the ethyl acetate extract and petroleum ether extract obtained by extraction using water, an alcohol (e.g., ethanol), or a water-alcohol (e.g., ethanol) mixture (e.g., 50-98% ethanol, such as 60% ethanol, 95% ethanol), and sequentially petroleum ether, chloroform, ethyl acetate, and n-butanol as the solvents. According to any aspect of the present invention, the extract is the ethyl acetate extract and comprises catechin, naringenin-4′-O-glucoside, (−)epicatechin-3-O-gallate, and phlorizin. According to any aspect of the present invention, the extract is the petroleum ether extract, and comprises chrysophanol. According to any aspect of the present invention, the extract is a mixture of the ethyl acetate extract and petroleum ether extract, and comprises catechin, naringenin-4′-O-glucoside, (−)epicatechin-3-O-gallate, phlorizin, chrysophanol. According to any aspect of the present invention, the extract is the ethyl acetate extract, petroleum ether extract, a mixture of the ethyl acetate extract and the petroleum ether extract, and comprises at least one of following ingredients selected from the group consisting of: chrysophanol, emodin, catechin, (−) epicatechin-3-O-gallate, naringenin-4′-O-glucoside, and phlorizin. According to any aspect of the present invention, the extract is the ethyl acetate extract, petroleum ether extract, or a mixture of ethyl acetate extract and the petroleum ether extract, and comprises at least one of ingredients selected from the group consisting of: chrysophanol, emodin, catechin, (−)epicatechin-3-β-gallate, naringenin-4′-O-glucoside, phlorizin, and the total amount of the chrysophanol, emodin, catechin, (−)epicatechin-3-O-gallate, naringenin-4′-O-glucoside and phlorizin relative to the total amount of the extraction is 1-90% (w/w), preferably 1-80% (w/w), preferably 2-50% (w/w), preferably 2-40% (w/w), preferably 2-30% (w/w), and more preferably 2-20% (w/w).

The Herba Cynomorii extract according to any embodiment of the second aspect of the present invention is prepared by a process comprising the following steps: extracting Herba Cynomorii with an aqueous ethanol solution (e.g., 50-98% ethanol, such as 60% ethanol, 95% ethanol); extracting the obtained alcohol extract with petroleum ether, and concentrating the ether layer to obtain a petroleum ether portion; extracting the residue of petroleum ether extraction with chloroform, and concentrating the chloroform layer to obtain a chloroform portion; extracting the residue of chloroform extraction with ethyl acetate, and concentrating the ethyl acetate layer to obtain an ethyl acetate portion; extracting the residue of ethyl acetate extraction with n-butanol, and concentrating the n-butanol layer to obtain a n-butanol portion; and combining the above petroleum ether portion, chloroform portion and ethyl acetate portion to obtain the extract. In an embodiment, the Herba Cynomorii extract is obtained by combining the above petroleum ether portion and chloroform portion. In an embodiment, said Herba Cynomorii extract is obtained by combining the above chloroform portion and ethyl acetate portion. In an embodiment, the Herba Cynomorii extract is obtained by combining the above petroleum ether portion and ethyl acetate portion. In an embodiment, the Herba Cynomorii extract is the above ethyl acetate portion.

The Herba Cynomorii extract according to any embodiment of the second aspect of the present invention is prepared by a process comprising the following steps: a) dipping Herba Cynomorii in 60-98% (preferably 70-98%, and more preferably 80-98%, such as 80%, 85%, 90%, 95% or 98%) ethanol at a volume of 2-20 times (preferably 5-15 times, and more preferably 8-12 times, such as 10 times) than the volume of Herba Cynomorii for 5-24 h (preferably 5-18 h, and more preferably 8-15 h); b) extracting under refluxing for 0.5-10 h (preferably 0.5-8 h, more preferably 0.5-6 h, more preferably 0.5-4 h, and further more preferably 1-4 h, such as 1 h, 2 h, 3 h, 4 h), and decanting the extract solution; c) optionally repeatedly subjecting the residue of step b) to step a) and step b) for 1-3 times (such as once, twice, 3 times); d) combining the extract solutions of step b) and step c), filtrating, and recovering ethanol from the filtrate to obtain a concentrated solution; and e) extracting the concentrated solution of step d) with petroleum ether, chloroform, ethyl acetate and n-butanol in order, recovering the solvents from each of the extracts to obtain a solid, and combining any one or more solids from the petroleum portion, chloroform portion and ethyl acetate portion to obtain the extract. In an embodiment, the Herba Cynomorii extract is obtained by combining the above petroleum portion and chloroform portion. In an embodiment, the Herba Cynomorii extract is obtained by combining the above chloroform portion and ethyl acetate portion. In an embodiment, the Herba Cynomorii extract is obtained by combining the above petroleum portion and the ethyl acetate portion. In an embodiment, the Herba Cynomorii extract is the above ethyl acetate portion.

The third aspect of the present invention provides one or more active ingredients selected from the group consisting of: chrysophanol, emodin, catechin, (−)epicatechin-3-β-gallate, naringenin-4′-O-glucoside, and phlorizin, which are obtainable from Herba Cynomorii. In an embodiment, the Herba Cynomorii active ingredients may be used for the treatment and/or prophylaxis of a disease associated with insufficient secretion of estrogens in a mammal (e.g., human). In an embodiment, the chrysophanol may be used for the treatment and/or prophylaxis of a disease associated with insufficient secretion of estrogens in a mammal (e.g., human). In an embodiment, the emodin may be used for the treatment and/or prophylaxis of a disease associated with insufficient secretion of estrogens in a mammal (e.g., human). In an embodiment, the catechin may be used for the treatment and/or prophylaxis of a disease associated with insufficient secretion of estrogens in a mammal (e.g., human). In an embodiment, the (−) epicatechin-3-O-gallate may be used for the treatment and/or prophylaxis of a disease associated with insufficient secretion of estrogens in a mammal (e.g., human). In an embodiment, the naringenin-4′-O-glucoside may be used for the treatment and/or prophylaxis of a disease associated with insufficient secretion of estrogens in a mammal (e.g., human). In an embodiment, the phlorizin may be used for the treatment and/or prophylaxis of a disease associated with insufficient secretion of estrogens in a mammal (e.g., human).

For the active ingredients according to any embodiment of the third aspect, one or more of the ingredients chrysophanol, emodin, catechin, (−)epicatechin-3-O-gallate, naringenin-4′-O-glucoside, and phlorizin are obtained from Herba Cynomorii via extraction.

For the active ingredients according to any embodiment of the third aspect of the present invention, the steps for extracting chrysophanol, emodin, catechin, (−)epicatechin-3-β-gallate, naringenin-4′-O-glucoside, and phlorizin from Herba Cynomorii are as follows:

extracting Herba Cynomorii with an aqueous ethanol solution (e.g., 50-98% ethanol, such as 60% ethanol, or 95% ethanol);

extracting the obtained alcohol extract with petroleum ether, chloroform, ethyl acetate and n-butanol in order, and recovering the solvent from each of the extracts respectively to obtain a solid of the extract for each of the extracting solvents;

subjecting the petroleum ether portion to a silica gel column chromatography, eluting with a gradient of petroleum ether-ethyl acetate solvent system, and subjecting the fractions 3-4 to recrystallization from petroleum ether to obtain chrysophanol;

subjecting the chloroform portion to a silica gel column chromatography, eluting with a gradient of petroleum ether-acetone solvent system, and subjecting the fractions 19-32 to a silica gel column chromatography with hydroxypropyl dextrane gel and a chloroform-methanol solvent system more times to obtain emodin;

subjecting the ethyl acetate portion to a silica gel column chromatography, eluting with a gradient of chloroform-methanol solvent system to obtain fractions 27-42 of the ethyl acetate portion, and fractions 43-64 of the ethyl acetate portion;

subjecting the fractions 27-42 of the ethyl acetate portion to a silica gel column chromatography, eluting with a gradient of chloroform-methanol-water solvent system to separately obtain fractions 27-42-4-6 (i.e., the fractions 4-6 obtained by subjecting the fractions 27-42 of the ethyl acetate portion from the above step to the silica gel column chromatography, similar expressions used herein in other places have a similar meaning), fractions 27-42-7-10, and fractions 27-42-11-14;

subjecting the fractions 27-42-4-6 to a silica gel column chromatography with a petroleum ether-ethyl acetate solvent system to obtain catechin;

subjecting the fractions 27-42-7-10 to a silica gel column chromatography and a reversed phase silica gel column chromatography to obtain naringenin-4′-O-glucoside;

subjecting the fractions 27-42-11-14 to a hydroxypropyl dextrane gel and a reversed phase silica gel column chromatography to obtain (−)epicatechin-3-O-gallate; and

subjecting the fractions 43-64 of the ethyl acetate portion to a polyamide and silica gel column chromatography with a chloroform-methanol solvent system in combination with a hydroxypropyl dextrane gel column chromatography to obtain phlorizin.

The fourth aspect of the present invention provides use of a Herba Cynomorii extract in the manufacture of a medicament for the treatment and/or prophylaxis of a disease associated with insufficient secretion of estrogens in a mammal (e.g., human).

According to the use of any embodiment of the fourth aspect of the present invention, the disease associated with insufficient secretion of estrogens is selected from the group consisting of: climacteric syndrome, osteoporosis, degenerative osteoporosis, cardiovascular and cerebrovascular diseases, and breast cancer.

According to the use of any embodiment of the fourth aspect of the present invention, the daily dose of the extract for the mammal relative to per kg of body weight is equivalent to 0.01-10 g, preferably 0.02-5 g, more preferably 0.05-1 g, and further more preferably 0.05-0.5 g of Herba Cynomorii raw material.

According to the use of any embodiment of the fourth aspect of the present invention, the extract comprises at least one of ingredients selected from the group consisting of: chrysophanol, emodin, catechin, (−)epicatechin-3-O-gallate, naringenin-4′-O-glucoside, and phlorizin. In an embodiment, the total amount of the chrysophanol, emodin, catechin, (−) epicatechin-3-O-gallate, naringenin-4′-O-glucoside, and phlorizin relative to the total weight of the extract is 1-90% (w/w), preferably 1-80% (w/w), preferably 2-50% (w/w), preferably 2-40% (w/w), preferably 2-30% (w/w), more preferably 2-20% (w/w).

According to the use of any embodiment of the fourth aspect of the present invention, in which the extract comprises at least one of the following ingredients selected from the group consisting of: catechin, (−)epicatechin-3-O-gallate, naringenin-4′-O-glucoside, and phlorizin. In an embodiment, the total amount of the catechin, (−)epicatechin-3-O-gallate, naringenin-4′-β-glucoside, phlorizin relative to the total amount of the extract is 1-90% (w/w), preferably 1-80% (w/w), preferably 2-50% (w/w), preferably 2-40% (w/w), preferably 2-30% (w/w), more preferably 2-20% (w/w).

According to the use of any embodiment of the fourth aspect of the present invention, the extract is an extract obtained from Herba Cynomorii raw material by using water, an alcohol (e.g., ethanol), or a water-alcohol (e.g., ethanol) mixture (e.g., 50-98% ethanol, such as 60% ethanol, 95% ethanol) as extracting solvent.

According to the use of any embodiment of the fourth aspect of the present invention, the extract is obtained by extraction using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol.

According to the use of any embodiment of the fourth aspect of the present invention, the extract is the petroleum ether extract, chloroform extract, ethyl acetate extract, or a mixture thereof, obtained by extraction using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol. In an embodiment, the extract is a mixture of petroleum ether extract, chloroform extract, and ethyl acetate extract obtained by extraction using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol. In an embodiment, the extract is a mixture of chloroform extract and ethyl acetate extract via extraction by using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol. In an embodiment, the extract is ethyl acetate extract obtained by extraction using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol. In an embodiment, the extract is petroleum ether extract obtained by extraction using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol. In an embodiment, the extract is a mixture of ethyl acetate extract and petroleum ether extract using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol.

According to the use of any embodiment of the fourth aspect of the present invention, the extract comprises at least one ingredients selected from the group consisting of: chrysophanol, emodin, catechin, (−)epicatechin-3-O-gallate, naringenin-4′-O-glucoside, and phlorizin. In an embodiment, the extract comprises the following ingredients: chrysophanol, emodin, catechin, (−)epicatechin-3-O-gallate, naringenin-4′-O-glucoside, and phlorizin.

According to the use of any embodiment of the fourth aspect of the present invention, the extract comprises at least one ingredients selected from the group consisting of: catechin, (−) epicatechin-3-O-gallate, naringenin-4′-O-glucoside, and phlorizin. In an embodiment, the extract comprises the following ingredients: catechin, (−)epicatechin-3-O-gallate, naringenin-4′-O-glucoside, and phlorizin. In an embodiment, the extract is ethyl acetate extract obtained by extraction using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol.

According to the use of any embodiment of the fourth aspect of the present invention, the Herba Cynomorii extract is prepared by a process comprising the following steps: extracting Herba Cynomorii with an aqueous ethanol solution (e.g., 50-98% ethanol, such as 60% ethanol, 95% ethanol); extracting the obtained alcohol extract with petroleum ether, and concentrating the ether layer to obtain a petroleum ether portion; extracting the residue of petroleum ether extraction with chloroform, and concentrating the chloroform layer to obtain a chloroform portion; extracting the residue of chloroform extraction with ethyl acetate, and concentrating the ethyl acetate layer to obtain an ethyl acetate portion; extracting the residue of ethyl acetate extraction with n-butanol, and concentrating the n-butanol layer to obtain a n-butanol portion; and combining the above petroleum ether portion, chloroform portion and ethyl acetate portion to obtain the extract. In an embodiment, the Herba Cynomorii extract is obtained by combining the above petroleum ether portion, chloroform portion. In an embodiment, the Herba Cynomorii extract is obtained by combining the above chloroform portion, ethyl acetate portion. In an embodiment, the Herba Cynomorii extract is obtained by combining the above petroleum ether portion, ethyl acetate portion. In an embodiment, the Herba Cynomorii extract is the above ethyl acetate portion.

According to the use of any embodiment of the fourth aspect of the present invention, the Herba Cynomorii extract is prepared by a process comprising the following steps: a) dipping Herba Cynomorii in 2-20 times (preferably 5-15 times, more preferably 8-12 times, such as 10 times) volume of 60-98% (preferably 70-98%, more preferably 80-98%, such as 80%, 85%, 90%, 95% or 98%) ethanol for 5-24 h (preferably 5-18 h, more preferably 8-15 h); b) extracting under refluxing for 0.5-10 h (preferably 0.5-8 h, more preferably 0.5-6 h, more preferably 0.5-4 h, more preferably 1-4 h, such as 1 h, 2 h, 3 h, 4 h), decanting the extract solution; c) optionally subjecting the residue of step b) to step a) and step b) repeatedly for 1-3 times (such as once, twice, 3 times); d) combining the extract solutions of step b) and step c), filtrating, recovering ethanol from filtrate to obtain a concentrated solution; e) subjecting the concentrated solution of step d) to petroleum ether, chloroform, ethyl acetate and n-butanol extraction in order, recovering the solvent from each of extracts to obtain solid, combining the solids of any one or more of petroleum ether portion, chloroform portion, and ethyl acetate portion to obtain the extract. In an embodiment, the Herba Cynomorii extract is obtained by combining the above petroleum ether portion, chloroform portion. In an embodiment, the Herba Cynomorii extract is obtained by combining the above chloroform portion, ethyl acetate portion. In an embodiment, the Herba Cynomorii extract is obtained by combining petroleum ether portion, ethyl acetate portion. In an embodiment, the Herba Cynomorii extract is the above ethyl acetate portion.

The fifth aspect of the present invention provides use of one or more ingredients in the manufacture of a medicament for the treatment and/or prophylaxis of a disease associated with insufficient secretion of estrogens in a mammal (e.g., human), in which the ingredients are selected from the group consisting of: chrysophanol, emodin, catechin, (−)epicatechin-3-β-gallate, naringenin-4′-O-glucoside, and phlorizin. In an embodiment, a use of chrysophanol in the manufacture of a medicament for the treatment and/or prophylaxis of a disease associated with insufficient secretion of estrogens in a mammal (e.g., human) is provided. In an embodiment, a use of emodin in the manufacture of a medicament for the treatment and/or prophylaxis of a disease associated with insufficient secretion of estrogens in a mammal (e.g., human) is provided. In an embodiment, a use of catechin in the manufacture of a medicament for the treatment and/or prophylaxis of a disease associated with insufficient secretion of estrogens in a mammal (e.g., human) is provided. In an embodiment, a use of (−)epicatechin-3-O-gallate in the manufacture of a medicament for the treatment and/or prophylaxis of a disease associated with insufficient secretion of estrogens in a mammal (e.g., human) is provided. In an embodiment, a use of naringenin-4′-O-glucoside in the manufacture of a medicament for the treatment and/or prophylaxis of a disease associated with insufficient secretion of estrogens in a mammal (e.g., human) is provided. In an embodiment, a use of phlorizin in the manufacture of a medicament for the treatment and/or prophylaxis of a disease associated with insufficient secretion of estrogens in a mammal (e.g., human) is provided. In an embodiment, one or more ingredients in the manufacture of a medicament for the treatment and/or prophylaxis of a disease associated with insufficient secretion of estrogens in a mammal (e.g., human) is provided, in which the ingredients are selected from the group consisting of: catechin, (−)epicatechin-3-β-gallate, naringenin-4′-O-glucoside, and phlorizin.

According to the use of any one item of the fifth aspect of the present invention, the disease associated with insufficient secretion of estrogens is selected from the group consisting of: climacteric syndrome, osteoporosis, degenerative osteoporosis, cardiovascular and cerebrovascular diseases, and breast cancer.

The use according to any one item of the fifth aspect of the present invention is the use of the following ingredients in the manufacture of a medicament for the treatment and/or prophylaxis of a disease associated with insufficient secretion of estrogens in a mammal (e.g., human), which ingredients are selected from the group consisting of: chrysophanol, emodin, catechin, (−)epicatechin-3-O-gallate, naringenin-4′-O-glucoside, and phlorizin.

According to the use of any one item of the fifth aspect of the present invention, one or more ingredients of chrysophanol, emodin, catechin, (−)epicatechin-3-O-gallate, naringenin-4′-O-glucoside, and phlorizin are obtained via extraction from Herba Cynomorii.

According to the use of any one item of the fifth aspect of the present invention, the steps for extracting chrysophanol, emodin, catechin, (−)epicatechin-3-O-gallate, naringenin-4′-β-glucoside, and phlorizin from Herba Cynomorii are as follows:

extracting Herba Cynomorii with an aqueous ethanol solution (e.g., 50-98% ethanol, such as 60% ethanol, 95% ethanol);

subjecting the obtained alcohol extract to petroleum ether, chloroform, ethyl acetate and n-butanol extraction in order, and recovering the solvent respectively from each of the extracts to obtain a solid of the extract for each of the extracting solvents;

subjecting the petroleum ether portion to a silica gel column chromatography, eluting with a gradient of petroleum ether-ethyl acetate solvent system, and subjecting the fractions 3-4 to recrystallization from petroleum ether to obtain chrysophanol;

subjecting the chloroform portion to a silica gel column chromatography, eluting with a gradient of petroleum ether-acetone solvent system, and subjecting repeatedly the fractions 19-32 to a silica gel column chromatography with hydroxypropyl dextrane gel and a chloroform-methanol solvent system to obtain emodin;

subjecting the ethyl acetate portion to a silica gel column chromatography, eluting with a gradient of chloroform-methanol solvent system to obtain fractions 27-42 of the ethyl acetate portion, and fractions 43-64 of the ethyl acetate portion;

subjecting the fractions 27-42 of the ethyl acetate portion to a silica gel column chromatography, eluting with a gradient of chloroform-methanol-water solvent system, to separately obtain fractions 27-42-4-6 (i.e., the fractions 4-6 obtained from the fractions 27-42 of the ethyl acetate portion from the last step by a silica gel column chromatography, similar expressions used herein in other places have a similar meaning), fractions 27-42-7-10, and fractions 27-42-11-14;

subjecting the fractions 27-42-4-6 to a silica gel column chromatography with a petroleum ether-ethyl acetate solvent system to obtain catechin;

subjecting the fractions 27-42-7-10 to a silica gel column chromatography and a reversed phase silica gel column chromatography to obtain naringenin-4′-O-glucoside;

subjecting the fractions 27-42-11-14 to a hydroxypropyl dextrane gel chromatography and a reversed phase silica gel column chromatography to obtain (−)epicatechin-3-O-gallate; subjecting the fractions 43-64 of the ethyl acetate portion to a polyamide and silica gel column chromatography with a chloroform-methanol solvent system in combination with a hydroxypropyl dextrane gel column chromatography to obtain phlorizin.

The sixth aspect of the present invention provides use of Herba Cynomorii in the manufacture of a medicament as phytoestrogen.

According to the use of any embodiment of the sixth aspect of the present invention, the medicament may be used for the treatment and/or prophylaxis of climacteric syndrome, osteoporosis, degenerative osteoporosis, cardiovascular and cerebrovascular diseases, and breast cancer.

According to the use of any embodiment of the sixth aspect of the present invention, the daily dose of the medicament for the mammal relative to per kg of body weight is equivalent to 0.01-10 g, preferably 0.02-5 g, more preferably 0.05-1 g, and further more preferably 0.05-0.5 g of Herba Cynomorii raw material.

According to the use of any embodiment of the sixth aspect of the present invention, the medicament comprises an extract obtained from Herba Cynomorii raw material.

According to the use of any embodiment of the sixth aspect of the present invention, the medicament comprises an extract obtained from Herba Cynomorii raw material via extraction by using water, an alcohol (e.g., ethanol), or a water-alcohol (e.g., ethanol) mixture (e.g., 50-98% ethanol, such as 60% ethanol, 95% ethanol) as the solvent.

According to the use of any embodiment of the sixth aspect of the present invention, the medicament comprises an extract from Herba Cynomorii raw material, which extract is obtained by extraction using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol.

According to the use of any embodiment of the sixth aspect of the present invention, the medicament comprises an extract from Herba Cynomorii raw material, which extract is the petroleum ether extract, chloroform extract, ethyl acetate extract, or a mixture thereof, obtained by extraction using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol. In an embodiment, the extract is a mixture of petroleum ether extract, chloroform extract, and ethyl acetate extract obtained by extraction using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol. In an embodiment, the extract is a mixture of chloroform extract and ethyl acetate extract obtained by extraction using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol. In an embodiment, the extract is ethyl acetate extract obtained by extraction using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol. In an embodiment, the extract is petroleum ether extract obtained by extraction using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol. In an embodiment, the extract is a mixture of ethyl acetate extract and petroleum ether extract via extraction by using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol.

According to the use of any embodiment of the sixth aspect of the present invention, the medicament comprises an extract from Herba Cynomorii raw material, which extract comprises at least one ingredients selected from the group consisting of: chrysophanol, emodin, catechin, (−)epicatechin-3-O-gallate, naringenin-4′-O-glucoside, and phlorizin. In an embodiment, the extract comprises the following ingredients: chrysophanol, emodin, catechin, (−) epicatechin-3-O-gallate, naringenin-4′-O-glucoside, and phlorizin.

According to the use of any embodiment of the sixth aspect of the present invention, the medicament comprises an extract from Herba Cynomorii raw material, which extract comprises at least one ingredients selected from the group consisting of: catechin, (−)epicatechin-3-O-gallate, naringenin-4′-O-glucoside, and phlorizin. In an embodiment, the extraction comprises the following ingredients: catechin, (−)epicatechin-3-O-gallate, naringenin-4′-O-glucoside, and phlorizin. In an embodiment, the extract is ethyl acetate extract obtained by extraction using water, an alcohol (e.g., ethanol), or a water-alcohol (e.g., ethanol) mixture (e.g., 50-98% ethanol, such as 60% ethanol, 95% ethanol) as the solvent, and sequentially petroleum ether, chloroform, ethyl acetate, and n-butanol.

According to the use of any embodiment of the sixth aspect of the present invention, the medicament comprises an extract from Herba Cynomorii raw material, which extract of Herba Cynomorii is prepared by a process comprising the following steps: extracting Herba Cynomorii with an aqueous ethanol solution (e.g., 50-98% ethanol, such as 60% ethanol, 95% ethanol); extracting the obtained alcohol extract with petroleum ether, and concentrating the ether layer to obtain a petroleum ether portion; extracting the residue of petroleum ether extraction with chloroform, and concentrating the chloroform layer to obtain a chloroform portion; extracting the residue of chloroform extraction with ethyl acetate, and concentrating the ethyl acetate layer to obtain an ethyl acetate portion; extracting the residue of ethyl acetate extraction with n-butanol, and concentrating the n-butanol layer to obtain a n-butanol portion; and combining the above petroleum ether portion, chloroform portion and ethyl acetate portion to obtain the extract. In an embodiment, the Herba Cynomorii extract is obtained by combining the above petroleum ether portion, chloroform portion. In an embodiment, the Herba Cynomorii extract is obtained by combining the above chloroform portion, ethyl acetate portion. In an embodiment, the Herba Cynomorii extract is obtained by combining the above petroleum ether portion, ethyl acetate portion. In an embodiment, the Herba Cynomorii extract is the above ethyl acetate portion.

In an embodiment of the use according to any embodiment of the sixth aspect of the present invention, the extract has the features of the extract of the first aspect, the second aspect or the third aspect of the present invention.

The seventh aspect of the present invention provides use of a Herba Cynomorii extract in the manufacture of a medicament as phytoestrogen.

According to the use of any embodiment of the seventh aspect of the present invention, the medicament may be used for the treatment and/or prophylaxis of climacteric syndrome, osteoporosis, degenerative osteoporosis, cardiovascular and cerebrovascular disease, and breast cancer.

According to the use of any embodiment of the seventh aspect of the present invention, the daily dose of the medicament for the mammal relative to per kg of body weight is equivalent to 0.01-10 g, preferably 0.02-5 g, more preferably 0.05-1 g, more preferably 0.05-0.5 g of Herba Cynomorii raw material.

According to the use of any embodiment of the seventh aspect of the present invention, the extract has the features of the extract according to any embodiment of the second aspect of the present invention.

The eighth aspect of the present invention provides use of one or more ingredients in the manufacture of a medicament as phytoestrogen: chrysophanol, emodin, catechin, (−) epicatechin-3-O-gallate, naringenin-4′-O-glucoside, and phlorizin.

According to the use of any embodiment of the eighth aspect of the present invention, the medicament may be used for the treatment and/or prophylaxis of climacteric syndrome, osteoporosis, degenerative osteoporosis, cardiovascular and cerebrovascular diseases, and breast cancer.

The use according to any embodiment of the eighth aspect of the present invention is a use of the following ingredients in the manufacture of a medicament as phytoestrogen: chrysophanol, emodin, catechin, (−)epicatechin-3-O-gallate, naringenin-4′-O-glucoside, and phlorizin.

According to the use of any embodiment of the eighth aspect of the present invention, the chrysophanol, emodin, catechin, (−)epicatechin-3-O-gallate, naringenin-4′-O-glucoside, phlorizin independently have the features of any embodiment of the third aspect of the present invention.

The ninth aspect of the present invention provides a pharmaceutical composition for the treatment and/or prophylaxis of a disease associated with insufficient secretion of estrogens in a mammal (e.g., human), which comprises a therapeutically and/or prophylactically effective amount of Herba Cynomorii extract and optionally a pharmaceutically acceptable excipient.

According to the pharmaceutical composition of any embodiment of the ninth aspect of the present invention, the Herba Cynomorii extract comprises at least one of ingredients selected from the group consisting of: chrysophanol, emodin, catechin, (−)epicatechin-3-β-gallate, naringenin-4′-O-glucoside, and phlorizin.

According to the pharmaceutical composition of any embodiment of the ninth aspect of the present invention, the Herba Cynomorii extract comprises the following ingredients: chrysophanol, emodin, catechin, (−)epicatechin-3-O-gallate, naringenin-4′-O-glucoside, and phlorizin.

According to the pharmaceutical composition of any embodiment of the ninth aspect of the present invention, the total amount of the chrysophanol, chrysophanol, emodin, catechin, (−)epicatechin-3-O-gallate, naringenin-4′-O-glucoside, and phlorizin relative to the total weight of the extract is 1-90% (w/w), preferably 1-80% (w/w), preferably 2-50% (w/w), preferably 2-40% (w/w), preferably 2-30% (w/w), and more preferably 2-20% (w/w).

According to the pharmaceutical composition of any embodiment of the ninth aspect of the present invention, the daily dose of the pharmaceutical composition administered to the mammal relative to per kg of body weight is equivalent to 0.01-10 g, preferably 0.02-5 g, more preferably 0.05-1 g, and further more preferably 0.05-0.5 g of Herba Cynomorii raw material.

According to the pharmaceutical composition of any embodiment of the ninth aspect of the present invention, the Herba Cynomorii extract is prepared by a process comprising the following steps: extracting with water, an alcohol (e.g., ethanol), or a water-alcohol (e.g., ethanol) mixture (e.g., 50-98% ethanol, such as 60% ethanol, 95% ethanol) as the solvent, and subjecting to petroleum ether, chloroform, ethyl acetate, and n-butanol extraction in order to obtain a petroleum ether extract, chloroform extract, ethyl acetate extract, or a mixture thereof.

According to the pharmaceutical composition of any embodiment of the ninth aspect of the present invention, the Herba Cynomorii extract has the features of the extract according to any embodiment of the second aspect of the present invention.

The tenth aspect of the present invention provides a pharmaceutical composition for the treatment and/or prophylaxis of a disease associated with insufficient secretion of estrogens in a mammal (e.g., human), which comprises a therapeutically and/or prophylactically effective amount of at least one pharmaceutically active ingredient and optionally a pharmaceutically acceptable excipient, wherein the pharmaceutically active ingredient is selected from chrysophanol, emodin, catechin, (−)epicatechin-3-O-gallate, naringenin-4′-O-glucoside, and phlorizin.

The pharmaceutical composition to any embodiment of the tenth aspect of the present invention, comprises a therapeutically and/or prophylactically effective amount of chrysophanol, emodin, catechin, (−)epicatechin-3-O-gallate, naringenin-4′-O-glucoside, phlorizin, and optionally a pharmaceutically acceptable excipient.

The pharmaceutical composition according to any embodiment of the tenth aspect of the present invention, comprises a therapeutically and/or prophylactically effective amount of catechin, (−)epicatechin-3-O-gallate, naringenin-4′-O-glucoside, phlorizin, and optionally a pharmaceutically acceptable excipient According to the pharmaceutical composition of any embodiment of the tenth aspect of the present invention, the chrysophanol, emodin, catechin, (−)epicatechin-3-O-gallate, naringenin-4′-O-glucoside, and phlorizin independently have the features of any embodiment of the third aspect of the present invention.

The eleventh aspect of the present invention provides a method for the treatment and/or prophylaxis of a disease associated with insufficient secretion of estrogens in a subject in need thereof, which comprises administering to the subject a therapeutically and/or prophylactically effective amount of Herba Cynomorii. In an embodiment, the Herba Cynomorii is administered to the subject in a form of medicament. In an embodiment, the medicament has the features of any embodiment of the first aspect of the present invention.

The eleventh aspect of the present invention further provides a method for the treatment and/or prophylaxis of a disease associated with insufficient secretion of estrogens in a subject in need thereof, which comprises administering to the subject a therapeutically and/or prophylactically effective amount of the Herba Cynomorii extract according to any embodiment of the second aspect of the present invention.

The eleventh aspect of the present invention further provides a method for the treatment and/or prophylaxis of a disease associated with insufficient secretion of estrogens in a subject in need thereof, which comprises administering to the subject a therapeutically and/or prophylactically effective amount of at least one of ingredients selected from the group consisting of: chrysophanol, emodin, catechin, (−)epicatechin-3-O-gallate, naringenin-4′-O-glucoside, and phlorizin. In an embodiment, the chrysophanol, emodin, catechin, (−)epicatechin-3-O-gallate, naringenin-4′-O-glucoside, and phlorizin independently have the features of any embodiment of the third aspect of the present invention.

According to the method of any embodiment of the eleventh aspect of the present invention, the disease associated with insufficient secretion of estrogens is selected from the group consisting of: climacteric syndrome, osteoporosis, degenerative osteoporosis, cardiovascular and cerebrovascular diseases, and breast cancer.

The features of any aspects of the present invention of any embodiment of the any aspects are also suitable for any other aspect or any embodiment of the any other aspect, as long as they are not contradictory between each other, and necessary modifications may be applied when they are compatible. In the present invention, for example, when “any embodiment of the first aspect of the present invention” is mentioned, the “any item” refers to any sub-aspect of the first aspect of the present invention; and similar expressions for other aspects have the same meanings.

The aspects and features of the present invention are further described as follows.

All references cited are incorporated herein in their entire by reference, and if any expressions in these documents have different meanings from those in the present invention, the expressions of the present invention should prevail. In addition, the terms and phrases used in the present invention have general meanings well known by those skilled in the art, unless specified and explained otherwise in the present invention, and when the mentioned terms and phrases have meanings inconsistent to their known meanings, the meanings described in the present invention should prevail.

The term “Herba Cynomorii” used herein meets the definition under entry “Herba Cynomorii” ( [symbol] ) of the First Part of the Pharmacopoeia of the People's Republic of China, 2005 Edition.

The term “about” used herein generally refers to allowable error range in the art, such as ±10%, such as ±5%, such as ±2%.

As used herein, the term “effective amount” refers to a dose that may fulfill treatment, prophylaxis, alleviation and/or relief of the disease or disorder in a subject as mentioned in the present invention.

As used herein, the term “pharmaceutical composition” may interchange for “composition”, and refers to a substance that may fulfill treatment, prophylaxis, alleviation and/or relief of the disease or disorder in a subject as mentioned in the present invention.

As used herein, the term “subject” or “patient” refers to an animal, especially a mammal, such as human, dog, monkey, cattle, horse, etc., which is administered with the composition and extract for treatment, prophylaxis, alleviation and/or relief of the disease or disorder as mentioned in the present invention.

As used herein, the term “disease or disorder” refers to a physical state of the subject, the physical state associates with the disease or disorder as mentioned in the present invention.

As used herein, unless specifically indicated otherwise, “%” generally refers to a weight/weight percentage when total matter is solid, and generally refers to a weight/volume percentage when total matter is liquid. Of course, when total matter is liquid and solute is liquid, the percentage for characterizing the liquid solute generally refers to volume/volume percentage. As for % used for describing ethanol, such as 95% ethanol, it has meanings well known in the art, for example, the definition in the guide section of the Second Part of Pharmacopoeia of the People's Republic of China, 2005 Edition.

The phrase “disease associated with insufficient secretion of estrogens” in the text has the meaning well known by those skilled in the art, and generally refers to diseases or disorders induced by insufficient secretion of estrogens in a female mammal, such as a human, especially, a climacteric woman, and these diseases or disorders include but are not limited to climacteric syndrome, osteoporosis, degenerative osteoporosis, cardiovascular and cerebrovascular diseases, and breast cancer.

As used herein, if not specifically indicated, the term “extract” refers to the “extract” of Herba Cynomorii.

As used herein, the term “catechin” may refer to (−)catechin, (+)catechin, or racemic catechin.

As used herein, the term “petroleum ether portion” refers to an effective site of plant obtained by petroleum ether treatment (extraction), and may also be called as “petroleum ether site”. Other similar terms such as “ethyl acetate portion” also have similar meanings, i.e., for example, effective site of plant obtained by liquid-liquid extraction.

Although the present invention describes the mentioned active ingredients in detail, the inventors of the present invention still further emphasize these active ingredients (which include but are not limited to chrysophanol, emodin, catechin, (−)epicatechin-3-O-gallate, naringenin-4′-O-glucoside, phlorizin) have chemical structure, physical and chemical properties as known by those skilled in the art.

According to the present invention, since 6 ingredients, chrysophanol, emodin, catechin, (−)epicatechin-3-O-gallate, naringenin-4′-O-glucoside, phlorizin, all have corresponding phytoestrogen functions, the amounts of these 6 ingredients or portions thereof in extract, medicament, composition (e.g., proportion of each of ingredients relative to total extract, medicament, composition, proportion between ingredients, and so on) are not specifically defined, and the dose of medicament may be calculated by conversion into Herba Cynomorii raw material.

A series of compounds obtained by separation are used in estrogen receptor activation test, in which estradiol is used as positive control, and all of chrysophanol, emodin, catechin, (−) epicatechin-3-O-gallate, naringenin-4′-O-glucoside, phlorizin show relevance to estrogen receptor activation.

Conventional techniques in pharmaceutical field may be used to mix single active ingredients of Herba Cynomorii or a combination thereof or an Herba Cynomorii extract containing estrogen active ingredients in Herba Cynomorii with one or more pharmaceutically acceptable excipients, and form a desired dosage form, so as to prepare the pharmaceutical composition of the present invention.

The term “pharmaceutically acceptable excipient” used in the pharmaceutical composition may be any conventional excipients in the pharmaceutical field. The selection of specific excipients depends on administration manner or disease type and state of a specific patient to be treated. Manufacture methods of pharmaceutical compositions suitable for specific administration manners are known in the art. For example, pharmaceutically acceptable excipients include conventional diluents, carriers, fillings, bindings, wetting agent, disintegrants, absorption enhancers, surfactants, absorption supports and lubricants in the pharmaceutical field. If necessary, flavors, preservatives and sweetening agents may be added to the pharmaceutical compositions.

The pharmaceutical composition of the present invention may be processed as tablets, powders granules, capsules, oral liquids, pastes, creams, or injection emulsions (aseptic powder for injection). The above dosage forms may be prepared according to conventional methods in the pharmaceutical field.

The studies show that the compounds obtained in the present invention have pseudo-estrogen function in different degree. Hence, it may be expected that the compound obtained in the present invention (e.g., chrysophanol, emodin, catechin, (−)epicatechin-3-O-gallate, naringenin-4′-O-glucoside, phlorizin) or the pharmaceutical composition comprising it may be used for treatment of climacteric syndrome, breast cancer and/or cardiovascular and cerebrovascular diseases. Or, the obtained in the present invention may be used for manufacture of a medicament for treatment of climacteric syndrome, breast cancer and/or cardiovascular and cerebrovascular diseases.

The present invention uses the cell transfection and luciferase reporter gene chemiluminescence detection methods to study the effects of ten single compounds from Herba Cynomorii on transcriptional activity of ERα and ERβ, indicating which ingredients of Herba Cynomorii have estrogen activity, and illustrating their target points and action routes. The results surprisingly show that four flavone compounds (catechin, phlorizin, naringenin-4′-β-glucoside and (−)epicatechin-3-O-gallate) have a potent activity of estrogen, both catechin (10⁻⁵M) and phlorizin (10⁻⁶-10⁻⁵M) may agonize the transcriptional activity of ERα and ERβ, and have no significant selectivity to receptor subtypes; naringenin-4′-O-glucoside (10⁻⁹-10⁻⁵M) may agonize the transcriptional activity of ERα and ERβ in a dose dependent manner, and has a higher selectivity to ERβ than ERα; while (−)epicatechin-3-O-gallate (10⁻⁸-10⁻⁵M) may agonize ERα and ERβ with the highest transcription activity at 10⁻⁷M, and has a higher selectivity to ERα than ERβ. The estrogen activity of the flavone compounds is about 1/1000- 1/10 of that of estradiol. The two anthraquinone compounds from Herba Cynomorii, chrysophanol and emodin may activate the receptor transcription activity at the high concentration of 10⁻⁶M, have no receptor selectivity, and their activity is about 1/1000- 1/100 of that of estradiol.

### EXEMPLARY MODES FOR CARRYING OUT THE INVENTION

The present invention is further illustrated by the following examples and experiments. It should be understood that these examples and experiments are merely provided for illustrating the invention, rather than restricting the invention in any way.

The materials and experimental methods used in the examples are generally and/or specifically described. Although many materials and methods used for fulfilling the present invention are well known in the art, they are still further provided with a description as detailed as possible. Those skilled in the art would clearly understand that the materials and methods used in the present invention in the text are known in the art, if they are not specifically described.

### Example 1

### Preparation of the Four Flavone Compounds and 2 Anthraquinone Compounds from Herba Cynomorii

1. Preparation of the Extract

40.0 kg of Herba Cynomorii raw material (purchased from Alxa, Inner Mongolia) was crushed into small pieces, added with 400 liters of 95% ethanol, and immersed overnight. After extracting under refluxing for 2 h, extract solution was decanted, and the residue was added with 400 liters of 60% ethanol and extracted under refluxing for 2 h. The extract solutions were combined and filtrated, and ethanol was recovered from the filtrate via rotatory evaporation. The residue obtained after concentrating was extracted sequentially with petroleum ether, chloroform, ethyl acetate and n-butanol, and a solid was obtained after recovering the solvents, respectively. Results: 55 g solid from the petroleum ether portion, 229 g solid from the chloroform portion, 147 g solid from the ethyl acetate portion, and 867 g solid from the n-butanol portion were obtained. The petroleum ether portion contained chrysophanol in a mass percentage of 0.082% (W/W), the chloroform portion contained emodin in 0.031% (W/W), the ethyl acetate portion contained catechin in a mass percentage of 10.84% (W/W), the ethyl acetate portion contained naringenin-4′-O-glucoside in a mass percentage of 0.304% (W/W), the ethyl acetate portion contained (−)epicatechin-3-O-gallate in a mass percentage of 0.88% (W/W), and the ethyl acetate portion contained phlorizin in a mass percentage of 0.28% (W/W).

2. Preparation of Individual Active Ingredients

55 g of the of the petroleum ether portion from Herba Cynomorii was subjected to a silica gel column chromatography, eluting with a gradient of petroleum ether-ethyl acetatesolvent system, and the fractions 3-4 were subjected to recrystallization from petroleum ether to obtain an orange red cluster crystal, 30 mg, identified as chrysophanol.

229 g of the chloroform portion of Herba Cynomorii was subjected to a silica gel column chromatography, eluting with a gradient of petroleum ether-acetone solvent system, the fractions 19-32 were subjected to a hydroxypropyl dextrane gel chromatography and repeated silica gel column chromatography with a chloroform-methanol solvent system to obtain orange red cluster crystal, 35 mg, identified as emodin.

147 g of the ethyl acetate portion Herba Cynomorii was subjected to a silica gel column chromatography, eluting with a gradient of chloroform-methanol solvent system to separately obtain fractions 27-42 of the ethyl acetate portion and fractions 43-64 of the ethyl acetate portion.

The fractions 27-42 of the ethyl acetate portion were subjected to a silica gel column chromatography, eluting with a gradient of chloroform-methanol-water solvent system. The obtained fractions 27-42-4-6 were subjected to a silica gel column chromatography with a petroleum ether-ethyl acetate solvent system to obtain a white cluster crystal, 13 g, identified as catechin (raceme). The obtained fractions 27-42-7-10 were subjected to a silica gel column chromatography and a reversed phase silica gel column chromatography to obtain white powder, 300 mg, identified as naringenin-4′-O-glucoside. The obtained fractions 27-42-11-14 were subjected to a hydroxypropyl dextrane gel chromatography and reversed phase silica gel column chromatography to obtain off-white powder, 1 g, identified as (−)epicatechin-3-O-gallate.

The fractions 43-64 of the ethyl acetate portion were subjected to a polyamide chromatography and silica gel column chromatography with a chloroform-methanol solvent system in combination with a hydroxypropyl dextrane gel column chromatography to obtain a brown needle crystal, 200 mg, identified as phlorizin.

### Example 2

### Confirmation of the Structures of the 6 Compounds from Herba Cynomorii

The structures of the 6 compounds (i.e., chrysophanol, emodin, catechin (raceme), (−)epicatechin-3-O-gallate, naringenin-4′-O-glucoside, phlorizin) obtained in Example 1 were confirmed (entrusting the Analysis Testing Center of Tianjin University to perform test, 500 MHz NMR). The results of analysis show that the structures of these compounds are consistent with those reported in the references, and the specific data are as follows.

Chrysophanol, as an orange red cluster crystal. ¹H NMR (CDCl₃, 500 MHz), δ: 7.09 (1H, br s, H-2), 7.64 (1H, br s, H-4), 7.81 (1H, br d, J=7.5 Hz, H-5), 7.67 (1H, br d, J=8.0 Hz, H-6), 7.28 (1H, br d, J=8.5 Hz, H-7), 2.47 (3H, s, —CH₃), 12.00 (1H, s, —OH), 12.12 (1H, s, —OH); ¹³C NMR (CDCl₃), δ: 162.6 (C-1), 124.8 (C-2), 149.6 (C-3), 121.6 (C-4), 124.6 (C-5), 137.1 (C-6), 120.1 (C-7), 162.9 (C-8), 192.7 (C-9), 182.1 (C-10), 133.8 (C-11), 113.9 (C-12), 116.0 (C-13), 133.5 (C-14), 22.5 (—CH₃).

Emodin, as an orange red cluster crystal. ¹H NMR (DMSO-d₆, 500 MHz), δ: 7.06 (1H, d, J=2 Hz, H-2), 7.39 (1H, d, J=2 Hz, H-4), 7.03 (1H, d, J=3.0 Hz, H-5), 6.50 (1H, d, J=3.0 Hz, H-7), 2.36 (3H, s, C6-CH₃).

Catechin (raceme), as a white cluster crystal. ¹H NMR (CD₃OD, 500 MHz), δ: 4.56 (1H, d, J=7.5 Hz, H-2), 3.97 (1H, m, H-3), 2.85 (1H, dd, J=16.0, 5.5 Hz, H-4a), 2.50 (1H, dd, J=16.0, 8.0 Hz, H-4b), 5.93 (1H, d, J=1.0 Hz, H-6), 5.86 (1H, br s, H-8), 6.84 (1H, br s, H-2′), 6.76 (1H, d, J=8.0 Hz, H-5′), 6.72 (1H, d, J=8.5 Hz, H-6′). ¹³C NMR (CD₃OD, 500 MHz), δ: 81.7 (C-2), 67.6 (C-3), 27.3 (C-4), 99.8 (C-4a), 156.4 (C-5), 95.1 (C-6), 156.6 (C-7), 94.3 (C-8), 155.7 (C-8a), 131.0 (C-1′), 114.1 (C-2′), 145.1 (C-3′), 145.1 (C-4′), 114.9 (C-5′), 118.9 (C-6′). The samples of 1.0 g/100 ml and 0.1 g/100 ml in methanol solution have specific optical rotation of zero.

(−)Epicatechin-3-O-gallate, as an off-white powder. ¹H NMR (CD₃OD, 500 MHz), δ: 5.03 (1H, s, H-2), 5.53 (1H, s, H-3), 2.99 (1H, dd, J=17.5, 4.5 Hz, H-4a), 2.85 (1H, dd, J=17.0, 1.5 Hz, H-4b), 5.96 (1H, s, H-6), 5.96 (1H, s, H-8), 6.93 (1H, d, J=2.0 Hz, H-2′), 6.70 (1H, d, J=7.5 Hz, H-5′), 6.81 (1H, dd, J=8.0, 2.0 Hz, H-6′), 6.95 (2H, s, H-2″ and H-6″). ¹³C NMR (CD₃OD, 500 MHz), δ: 78.5 (C-2), 69.9 (C-3), 26.8 (C-4), 99.4 (C-4a), 157.7 (C-5 and C-7), 96.5 (C-6), 95.8 (C-8), 157.2 (C-8a), 131.4 (C-1′), 115.0 (C-2′), 145.8 (C-3′ and C-4′), 116.0 (C-5′), 119.3 (C-6′), 121.3 (C-1″), 110.1 (C-2″ and C-6″), 146.2 (C-3″ and C-5″), 139.7 (C-4″), 167.6 (C—CO).

Naringenin-4′-O-glucoside, as a white powder. ¹H-NMR (DMSO-d₆, 500 MHz), δ: 5.52 (1H, dd, J=13, 3.0 Hz, H-2), 2.72-3.20 (2H, m, H-3), 5.88 (1H, d, J=1.5 Hz, H-6), 5.87 (1H, d, J=2.0 Hz, H-8), 7.42 (2H, d, J=8.5 Hz, H-2′ and H-6′), 7.05 (2H, d, J=9.0 Hz, H-3′ and H-5′), 4.88 (1H, d, J=7.5 Hz, saccharide H-1), 3.17-4.55 (6H, m, saccharide proton), 12.13 (1H, s, C₅—OH).

Phlorizin, as a brown needle crystal. ¹H NMR (CD₃OD, 500 MHz), δ: 7.06 (2H, d, J=8.5 Hz, H-2, 6), 6.68 (2H, d, J=8.5 Hz, H-3, 5), 6.18 (1H, d, J=2.0 Hz, H-3′), 5.95 (1H, d, J=2.0 Hz, H-5′), 5.04 (1H, d, J=7.0 Hz, H-1″), 3.43-3.48 (3H, overlapped, H-2″, 3″, 5″), 3.43 (1H, m, H-4″), 3.91 (1H, d, J=2.0, 12.5 Hz, H-6a″), 3.72 (1H, d, J=5.5, 12.5 Hz, H-6b″), 3.40 (2H, t, J=7.5 Hz, H-α), 2.88 (2H, t, J=7.5 Hz, H-β). ¹³C NMR (CD₃OD, 500 MHz), δ: 133.8 (C-1), 130.4 (C-2, 6), 116.1 (C-3, 5), 156.3 (C-4), 46.9 (C-α), 30.8 (C-β), 106.8 (C-1′), 162.3 (C-2′), 95.4 (C-3′), 165.9 (C-4′), 98.3 (C-5′), 167.5 (C-6′), 102.2 (C-1″), 74.7 (C-2″), 78.4 (C-3″), 71.1 (C-4″), 78.4 (C-5″), 62.4 (C-6″).

### Example 3

### Activation of the Compounds on the Estrogen Receptors

Cell transfection method was used to observe the selective activation of estrogen receptor by catechin, (−)epicatechin-3-O-gallate, naringenin-4′-O-glucoside, phlorizin, emodin, chrysophanol. Actually, estrogens show their biological activity by regulating estrogen responding genetic transcription.

The reporter gene-carrying ER_{α}, or ER_{β}DNA fragments were separately transfected in ER expression free Hela cells, after the substance to be tested was added to cell culture medium, the reporter gene transcription activation was observed to determine whether the substance to be tested exerts estrogen function via ER_{α}, or ER_{β}.

Formulation of sample solutions of catechin, (−)epicatechin-3-O-gallate, naringenin-4′-O-glucoside, phlorizin, emodin, chrysophanol (separately obtained from Example 1):

Catechin, (−)epicatechin-3-O-gallate, naringenin-4′-O-glucoside, phlorizin, emodin, chrysophanol in corresponding weights were separately weighed precisely, than added with DMSO for dissolution, the corresponding sample solution high dose groups (10 μM or 1 μM), were diluted in certain times to separately obtain low dose groups (1 μM to 0.001 μM).

Formulation of solutions of ethyl acetate portion and petroleum ether portion: Ethyl acetate portion (from 147 g of production obtained in Example) and petroleum ether portion (from 55 g of production obtained in Example) of Herba Cynomorii were separately weighed precisely and added with corresponding weights of DMSO for dissolution, the corresponding sample solution high dose group (50 μg/ml) were diluted in certain times to separately obtain low dose groups (5 μg/ml, 0.5 μg/ml and 0.05 μg/ml).

Under condition of 37° C. and 5% CO₂, Hela cells were cultured in phenol red-free high sugar DMEM culture medium containing 10% calf serum in incubator, the cells were spread in 60% density on 96-well plate when cell density was about 90%, each well was added with 150 ul of phenol red-free DMEM culture medium containing 10% serum, cultivation was performed overnight. To each well of the 96-well plate, 100 ul of serum-free DMEM culture medium and 0.4 μg plasmid (ptk-ERE-luc 2 μg, tk-Renila 1 μg, ERα or ERβ 1 μg) were used for transfection. After 6 h of transfection, each well was added with 150 ul of serum-free phenol red-free DMEM culture medium. Three wells were selected form each group, added with 10⁻⁸M estradiol E₂, and substance to be tested in different concentrations, 15 ul/well (sample 4-5 concentration gradient groups), continuously cultured under condition of 37° C., 5% CO₂ for 24 h. After 24 h, each well was added with 80 μl of cell disruption solution, and the cells were subjected to freeze thawing once to be sufficiently disrupted. As for the cell lysis solution of each well, its activities to firefly luciferase and Renila luciferase were separately measured. Each well used firefly luciferase/Renila luciferase value as result, the well without estrogen treatment in each group was used as control, the ratio of the group added with estrogen or substance to be tested to the control group was calculated, and the average value of three wells of each group was used. The results are shown in Table 1, Table 2 and Table 3.

TABLE 1
Effects of the petroleum ether portion and ethyl acetate portion from
Herba Cynomorii on ERα or ERβ transcription activity in Hela cells

| | Con- | ERα (% of the control) | ERβ (% of the control) |
|---|---|---|---|
| | centration | mean ± S.D. | mean ± S.D. |
| Sample | (μg/ml) | (% of estradiol) | (% of estradiol) |
| Ethyl | 50 | 94.33 ± 5.80 (44.92) | 119.21 ± 1.86 (51.68) |
| acetate | 5 | 90.83 ± 7.34 (43.25) | 118.94 ± 5.63 (49.56) |
| portion | 0.5 | 84.18 ± 12.84 (40.08) | 115.32 ± 2.46 (48.05) |
| | 0.05 | 79.275 ± 8.57 (37.75) | 94.42 ± 2.46 (39.34) |
| Petroleum | 50 | 73.51 ± 8.76 (39.10) | 75.99 ± 12.83 (38.97) |
| ether | 5 | 75.09 ± 9.87 (39.94) | 59.09 ± 15.27 (30.30) |
| portion | 0.5 | 54.42 ± 9.89 (28.95) | 71.67 ± 11.31 (36.76) |
| | 0.05 | 65.64 ± 6.76 (34.92) | 75.99 ± 5.91 (38.97) |

The results in the table show that the extracts of the present invention have the desired biological activity. Those skilled in the art would understand that although only the activity of the ethyl acetate portion and petroleum ether portion are indicated in the table, it may be expected that any one or more of the extracts obtained before the extraction with petroleum ether, chloroform, ethyl acetate and n-butanol and the effective portions obtained after the extraction with the four solvents would have the desired biological activity.

TABLE 2
Effects of the flavone compounds on ERα or ERβ transcription activity in Hela cells

| | | | ERβ (% of the | |
|---|---|---|---|---|
| | | ERα (% of the control) | control) | ERβ/ERα |
| | Concentration | mean ± S.D. | mean ± S.D. | (both % of |
| Sample | (M) | (% of estradiol) | (% of estradiol) | estradiol at 10⁻⁸M) |
| Catechin | 10⁻⁸ | 102.89 ± 10.21 | 101.93 ± 2.27 | 1.09 |
| | | (48.42) | (52.79) | |
| | 10⁻⁷ | 106.72 ± 12.84 | 102.57 ± 1.04 | 1.06 |
| | | (50.22) | (53.12) | |
| | 10⁻⁶ | 108.85 ± 8.87 | 103.71 ± 9.07 | 1.05 |
| | | (51.22) | (53.71) | |
| | 10⁻⁵ | 128.00 ± 8.74** | 115.59 ± 13.39* | 1.28 |
| | | (60.23) | (59.86) | |
| (−)Epicatechin- | 10⁻⁹ | 121.95 ± 10.76 | 97.75 ± 17.66 | 0.94 |
| | | (57.32) | (53.82) | |
| 3-O-gallate((−)- | 10⁻⁸ | 170.00 ± 13.87** | 131.16 ± 15.95* | 0.97 |
| Epicatechin | | (79.90) | (77.72) | |
| gallate) | 10⁻⁷ | 200.50 ± 13.72** | 159.55 ± 28.00** | 0.93 |
| | | (94.24) | (87.84) | |
| | 10⁻⁶ | 192.40 ± 27.72** | 148.13 ± 4.22** | 0.90 |
| | | (90.43) | (81.56) | |
| | 10⁻⁵ | 171.88 ± 22.57** | 137.80 ± 15.93** | 0.94 |
| | | (80.79) | (75.87) | |
| Naringenin- | 10⁻⁹ | 110.04 ± 5.59 | 137.90 ± 9.40** | 1.29 |
| 4′-O- | | (53.76) | (69.45) | |
| glucoside | 10⁻⁸ | 157.62 ± 14.54** | 180.98 ± 10.46** | 1.18 |
| (Naringenin- | | (77.00) | (91.14) | |
| 4′-0-β-D- | 10⁻⁷ | 182.34 ± 5.97** | 200.66 ± 5.22** | 1.13 |
| glucopyranoside) | | (89.08) | (101.06) | |
| | 10⁻⁶ | 198.72 ± 3.99** | 207.11 ± 15.76** | 1.07 |
| | | (97.08) | (104.30) | |
| | 10⁻⁵ | 188.07 ± 18.61** | 215.85 ± 22.32** | 1.18 |
| | | (91.88) | (108.71) | |
| Phlorizin | 10⁻⁸ | 105.43 ± 13.57 | 109.38 ± 8.72 | 1.07 |
| (Phlorizin) | | (55.41) | (59.27) | |
| | 10⁻⁷ | 118.40 ± 4.20 | 116.14 ± 8.86 | 1.01 |
| | | (62.23) | (62.93) | |
| | 10⁻⁶ | 122.09 ± 11.33* | 133.79 ± 5.45** | 1.13 |
| | | (64.17) | (72.50) | |
| | 10⁻⁵ | 137.71 ± 8.44** | 147.95 ± 10.29** | 1.11 |
| | | (72.38) | (80.17) | |

In the table, *P < 0.05, **P < 0.01, in comparison with the control

TABLE 3
Effects of the anthraquinone compounds on ERα or ERβ transcription activity in Hela cells

| | | ERα (% of the | ERβ (% of the | |
|---|---|---|---|---|
| | | control) | control) | ERβ/ERα |
| | Concentration | mean ± S.D. | mean ± S.D. | (both % of estradiol |
| Sample | (M) | (% of estradiol) | (% of estradiol) | at 10⁻⁸M) |
| Chrysophanol | 10⁻⁹ | 84.53 ± 14.21 | 86.99 ± 23.16 | 1.25 |
| | | (35.43) | (44.25) | |
| (Chrysophanol) | 10⁻⁸ | 80.15 ± 13.88 | 76.85 ± 8.82 | 1.16 |
| | | (33.59) | (39.09) | |
| | 10⁻⁷ | 97.77 ± 8.47 | 83.12 ± 4.78 | 1.03 |
| | | (40.98) | (42.28) | |
| | 10⁻⁶ | 122.89 ± 8.94** | 120.43 ± 12.98** | 1.19 |
| | | (51.51) | (61.26) | |
| Emodin | 10⁻⁹ | 101.27 ± 15.90 | 104.68 ± 13.96 (47.31) | 1.07 |
| (Emodin) | | (44.27) | | |
| | 10⁻⁸ | 108.48 ± 27.45 | 104.92 ± 9.08 | 1.00 |
| | | (47.43) | (47.42) | |
| | 10⁻⁷ | 109.96 ± 11.33 | 106.60 ± 10.76 | 1.00 |
| | | (48.07) | (48.18) | |
| | 10⁻⁶ | 146.40 ± 18.07** | 145.21 ± 8.06** | 1.03 |
| | | (64.01) | (65.63) | |

In the table, *P < 0.05, **P < 0.01, in comparison with the control

Estrogens mainly exert their effects through estrogen receptor (ER). The studies confirm that ER widely exists incardiovascular system, osteoblasts, osteoclasts, mammary gland, and reproductive system, estrogens therefore have wide therapeutical effects, while phytoestrogen is a natural selective estrogen receptor regulating agent, which has similar structure and function with estrogens, may bind to restrogen receptor and has also estrogen like biological activity. The studying results show that the compounds of the present invention may activate estrogen receptor in concentration dependent manner. Our studies also confirm many ingredients in Herba Cynomorii may bind to ER, increase the transcription activity thereof, thereby confirming they also has estrogen like functions. It also suggests that the catechin, epicatechingallate, naringeninglucoside, phlorizin, emodin, chrysophanol in Herba Cynomorii may be used for treatment of climacteric syndrome of women, osteoporosis, degenerative osteoporosis, cardiovascular and cerebrovascular diseases.

The present invention is not limited to the above examples, and those skilled in the art may obtain other modified embodiments of the present invention without departing from the spirit and scope of the present invention.

What is claimed is:

1. Use of Herba Cynomorii in the manufacture of a medicament for the treatment and/or prophylaxis of a disease associated with insufficient secretion of estrogens in a mammal.

2. The use according to claim 1, wherein the disease associated with insufficient secretion of estrogens is selected from the group consisting of: climacteric syndrome, osteoporosis, degenerative osteoporosis, cardiovascular and cerebrovascular diseases, and breast cancer.

3. The use according to claim 1, wherein the medicament comprises an extract from Herba Cynomorii raw material obtained by using water, an alcohol, or a water-alcohol mixture as the extracting solvent.

4. The use according to claim 1, wherein the medicament comprises an extract from Herba Cynomorii raw material, which extract comprises at least one of ingredients selected from the group consisting of: chrysophanol, emodin, catechin, (−)epicatechin-3-O-gallate, naringenin-4′-O-glucoside, and phlorizin.

5. An extract from Herba Cynomorii, which comprises at least one of ingredients selected from the group consisting of: chrysophanol, emodin, catechin, (−)epicatechin-3-β-gallate, naringenin-4′-O-glucoside, and phlorizin.

6. The extract from Herba Cynomorii according to claim 5, which is obtained by extraction using water, an alcohol or a water-alcohol mixture as the solvent, and sequentially petroleum ether, chloroform, ethyl acetate, and n-butanol.

7. Use of an extract from Herba Cynomorii in the manufacture of a medicament for the treatment and/or prophylaxis of a disease associated with insufficient secretion of estrogens in a mammal.

8. The use according to claim 7, wherein the disease associated with insufficient secretion of estrogens is selected from the group consisting of: climacteric syndrome, osteoporosis, degenerative osteoporosis, cardiovascular and cerebrovascular diseases, and breast cancer.

9. The use according to claim 7, wherein the extract comprises at least one of ingredients selected from the group consisting of: chrysophanol, emodin, catechin, (−) epicatechin-3-O-gallate, naringenin-4′-O-glucoside, and phlorizin.

10. The use according to claim 7, wherein the extract is an extract obtained from Herba Cynomorii raw material by using water, an alcohol, or a water-alcohol mixture as the extracting solvent.

11. The use according to claim 7, wherein the extract from Herba Cynomorii is prepared by a process comprising the following steps: extracting Herba Cynomorii with an aqueous ethanol solution; extracting the obtained alcohol extract with petroleum ether, and concentrating the ether layer to obtain a petroleum ether portion; extracting the residue of petroleum ether extraction with chloroform, and concentrating the chloroform layer to obtain a chloroform portion; extracting the residue of chloroform extraction with ethyl acetate, and concentrating the ethyl acetate layer to obtain an ethyl acetate portion; extracting the residue of ethyl acetate extraction with n-butanol, and concentrating the n-butanol layer to obtain a n-butanol portion; and combining the above petroleum ether portion, chloroform portion and ethyl acetate portion to obtain the extract.

12. Use of one or more ingredients selected from the group consisting of chrysophanol, emodin, catechin, (−)epicatechin-3-O-gallate, naringenin-4′-O-glucoside, and phlorizin in the manufacture of a medicament for the treatment and/or prophylaxis of a disease associated with insufficient secretion of estrogens in a mammal.

13. The use according to claim 12, wherein the disease associated with insufficient secretion of estrogens is selected from the group consisting of: climacteric syndrome, osteoporosis, degenerative osteoporosis, cardiovascular and cerebrovascular diseases, and breast cancer.

14. Use of Herba Cynomorii in the manufacture of a medicament as phytoestrogen.

15. Use of an extract from Herba Cynomorii in the manufacture of a medicament as phytoestrogen.

16. Use of one or more ingredients selected from the group consisting of chrysophanol, emodin, catechin, (−)epicatechin-3-O-gallate, naringenin-4′-O-glucoside, and phlorizin in the manufacture of a medicament as phytoestrogen.

17. A pharmaceutical composition for the treatment and/or prophylaxis of a disease associated with insufficient secretion of estrogens in a mammal comprising a therapeutically and/or prophylactically effective amount of an extract from Herba Cynomorii, and optionally a pharmaceutically acceptable excipient.

18. A pharmaceutical composition for the treatment and/or prophylaxis of a disease associated with insufficient secretion of estrogens in a mammal comprising a therapeutically and/or prophylactically effective amount of at least one pharmaceutically active ingredient and optionally a pharmaceutically acceptable excipient, wherein the pharmaceutically active ingredient is selected from the group consisting of: chrysophanol, emodin, catechin, (−) epicatechin-3-O-gallate, naringenin-4′-O-glucoside, and phlorizin.

19. A method for the treatment and/or prophylaxis of a disease associated with insufficient secretion of estrogens in a subject in need thereof comprising administering to the subject an effective amount of Herba Cynomorii, an extract from Herba Cynomorii, or at least one of ingredients selected from the group consisting of: chrysophanol, emodin, catechin, (−) epicatechin-3-O-gallate, naringenin-4′-O-glucoside, and phlorizin.

20. The method according to claim 20, wherein the disease associated with insufficient secretion of estrogens is selected from the group consisting of: climacteric syndrome, osteoporosis, degenerative osteoporosis, cardiovascular and cerebrovascular diseases, and breast cancer.
